(19) United States
(12) Patent Application Publication
(10) Pub. No.: US 2023/0407351 A1
(43) Pub. Date: Dec. 21, 2023

(54) RECOMBINANT HOST CELLS TO PRODUCE ANTHRANILIC ACID

(71) Applicant: PILI, TOULOUSE CEDEX 4 (FR)

(72) Inventors: LUCIE CREPIN, TOULOUSE CEDEX 4 (FR); CAROLINE SCHIAVON, TOULOUSE CEDEX 4 (FR)

(21) Appl. No.: 18/250,989

(22) PCT Filed: Oct. 28, 2021
(86) PCT No.: PCT/EP2021/079927

§ 371 Date: Apr. 28, 2023

(30) Foreign Application Priority Data
Oct. 28, 2020 (EP) 20306293.0

(51) Int. Cl. C12P 13/04; C12N 15/70; C12N 9/10; C12N 9/88; C12N 9/90; C12N 9/00

(52) U.S. Cl. CPC C12P 13/04; C12N 15/70; C12N 9/1022; C12Y 202/01001; C12N 9/1077; C12Y 204/02018; C12N 9/88; C12Y 401/01048; C12Y 402/0102; C12N 9/90; C12Y 503/01024; C12N 9/93; C12Y 603/05002; C12N 2800/101

(57) ABSTRACT

The present invention relates to a recombinant bacterium genetically modified to produce anthranilic acid and being able to grow in a culture medium lacking tryptophan. It also relates to a method for producing anthranilic acid using said recombinant bacterium.

### FIELD OF THE INVENTION

The present invention relates to the field of the production of anthranilic acid, in particular the production of anthranilic acid using recombinant host cells.

### BACKGROUND OF THE INVENTION

Anthranilic acid is a natural intermediate of the shikimic acid pathway and a precursor for the biosynthesis of the aromatic amino acid L-tryptophane. It is industrially used as an intermediate for the synthesis of dyes, perfumes, pharmaceuticals and other valuable products.

Chemical synthesis of anthranilate is an unsustainable and expensive process requiring conditions of high temperature and pressure and nonrenewable naphtalene and generating toxic by-products. There is thus a strong need for sustainable technologies providing renewable and biologically derived source of anthranilate.

Some microorganisms and plants have the metabolic capacity to synthesize anthranilate. In particular, the biosynthetic pathway of anthranilic acid and aromatic acids in bacteria is quite well understood. As illustration, FIG. 1 represents a simplified scheme of this pathway in Escherichia coli. Briefly, the first intermediate, 3 deoxy-D-arabino-heptulosenate-7-phosphaste, is produced from two precursors, Erythrose 4 phosphate (E4P) from pentose phosphate pathway and phosphoenolpyruvate (PEP) from glycolysis, by DAHP synthases AroF, AroH or AroG. Then aroB, aroD and aroE catalyze three reactions from DAHP to produce shikimic acid (SHK). Subsequently, chorismic acid is synthetized from SHK by AroL/K, AroA and AroC. Eventually, TrpE and TrpG catalyze the production of anthranilic acid from chorismic acid. Anthranilic acid is then used to produce tryptophan after several reactions catalyzed by enzymes TrpD, TrpC, TrpA and TrpB successively. Thus, as a metabolic intermediate, anthranilic acid does not accumulate and is not secreted.

Early studies on the L-Tryptophan operon in E. coli identified mutants that secreted anthranilate (Yanofsky et al., Genetics, 1971, 69, 409-433). The characterization of one of the strains (W3110 trpD9923) obtained by UV mutagenesis revealed that the mutation was present in the gene encoding the bifunctional protein TrpGD, leading to stop codon and non-functional TrpD domain. In an attempt to provide microbial strains for anthranilate production, this strain was further modified to overexpress genes encoding a feedback inhibition resistant DAHP synthase, transketolase, glucokinase and galactose permease (Balderas-Hernindez et al. Microb Cell Fact. 2009 Apr. 2; 8:19). Similarly, an anthranilate accumulation was also observed in a mutant Bacillus strain (U.S. Pat. No. 5,422,256).

However, as a major drawback, these strains are tryptophan auxotrophs. Using auxotroph strains results in a dramatic increase of the costs in industrial production due to the need of adding tryptophan in the culture medium. Consequently, there is still a strong need for a much improved process achieving industrially relevant productivity of anthranilic acid using tryptophan non-auxotroph bacterial strains.

### SUMMARY OF THE INVENTION

The inventors aim at the production of anthranilic acid in a recombinant bacterial host cell which is a tryptophan non-auxotroph strain, i.e. a strain which is able to grow in a culture medium lacking tryptophan.

Accordingly, in a first aspect, the present invention relates to a method for producing anthranilic acid comprising culturing a recombinant bacterium which has been genetically modified to induce an imbalance between anthranilate phosphoribosyltransferase activity and anthranilate synthase activity in favor of anthranilate synthase activity, and is able to grow in a culture medium lacking tryptophan, and optionally, recovering anthranilic acid. In a preferred embodiment, the non-modified bacterium corresponding to the recombinant bacterium, expresses a bifunctional protein exhibiting TrpG and TrpD activities and the recombinant bacterium has been genetically modified to separately express a polypeptide exhibiting glutamine amidotransferase activity (TrpG) and a polypeptide exhibiting anthranilate phosphoribosyltransferase activity (TrpD).

In particular, the present invention relates to a method for producing anthranilic acid comprising culturing a recombinant bacterium and optionally, recovering anthranilic acid, wherein the non-modified bacterium corresponding to said recombinant bacterium expresses a bifunctional protein exhibiting TrpG and TrpD activities and the recombinant bacterium has been genetically modified to separately express (i) a polypeptide which exhibits glutamine amidotransferase activity (TrpG) and does not exhibit anthranilate phosphoribosyltransferase activity, and (ii) a polypeptide which exhibits anthranilate phosphoribosyltransferase activity (TrpD) and does not exhibit glutamine amidotransferase activity, and to decrease the ratio between anthranilate phosphoribosyltransferase activity and anthranilate synthase activity, by comparison to the non-modified bacterium, said recombinant bacterium being able to grow in a culture medium lacking tryptophan.

Preferably, the polypeptide which exhibits glutamine amidotransferase activity (TrpG) does not exhibit anthranilate phosphoribosyltransferase activity and the polypeptide which exhibits anthranilate phosphoribosyltransferase activity (TrpD) does not exhibit glutamine amidotransferase activity.

The recombinant bacterium may have been genetically modified to suppress expression of the TrpD domain of the endogenous bifunctional protein TrpGD, preferably by deleting all or part of the nucleic acid sequence encoding said domain.

Preferably, the recombinant bacterium has been further genetically modified to suppress expression of the endogenous bifunctional protein TrpGD, preferably by deleting all or part of the nucleic acid sequence encoding said protein.

In particular, the bacterium may be Escherichia coli.

The recombinant bacterium may comprise (i) a recombinant nucleic acid comprising a gene encoding glutamine amidotransferase (TrpG) or a first operon, under the control of a first promoter, wherein said first operon comprises at least a nucleic acid sequence encoding TrpG and optionally a nucleic acid sequence encoding anthranilate synthase (TrpE), and/or (ii) a recombinant nucleic acid comprising a gene encoding anthranilate phosphoribosyltransferase (TrpD) or a second operon, under the control of a second promoter wherein said second operon comprises at least a nucleic acid sequence encoding TrpD. In some particular embodiments, the first promoter is stronger than the second promoter. Preferably, the first promoter is a promoter that is not operably linked to a gene encoding TrpG in the non-modified bacterium and/or the second promoter is a promoter that is not operably linked to a gene encoding TrpD in the non-modified bacterium. The second operon may further comprise a nucleic acid sequence encoding indole-3-glycerol phosphate synthase enzyme (TrpC), a nucleic acid sequence encoding phosphoribosylanthranilate isomerase enzyme (TrpF), a nucleic acid sequence encoding α subunit of tryptophan synthase enzyme (TrpA) and/or a nucleic acid sequence encoding β subunit of tryptophan synthase enzyme (TrpB).

Alternatively, the recombinant bacterium may comprise a gene encoding glutamine amidotransferase (TrpG) and a gene encoding anthranilate phosphoribosyltransferase (TrpD), under the control of the same promoter thereby expressing two distinct proteins TrpG and TrpD.

The recombinant bacterium may also be genetically modified to express a gene encoding a feedback resistant 3-deoxy-D-arabino-heptulosonate-7-phosphate synthase enzyme and/or genetically modified to overexpress an endogenous gene encoding a transketolase enzyme or to express a heterologous gene encoding a transketolase enzyme.

Preferably, in the method of the invention, the recombinant bacterium is cultivated in a culture medium lacking tryptophan or any tryptophan source.

In another aspect, the present invention relates to a recombinant bacterium as defined in above. In particular, the present invention relates to a recombinant Escherichia coli bacterium which has been genetically modified to comprise a nucleic acid sequence encoding glutamine amidotransferase (TrpG) under the control of a first promoter and a nucleic acid sequence encoding anthranilate phosphoribosyltransferase (TrpD) under the control of a second promoter. Preferably, said recombinant bacterium comprises (i) a gene encoding glutamine amidotransferase (TrpG) or a first operon, under the control of a first promoter, wherein said first operon comprises at least a nucleic acid sequence encoding TrpG and optionally a nucleic acid sequence encoding anthranilate synthase (TrpE), and (ii) a gene encoding anthranilate phosphoribosyltransferase (TrpD) or a second operon, under the control of a second promoter wherein said second operon comprises at least a nucleic acid sequence encoding TrpD, and optionally a nucleic acid sequence encoding indole-3-glycerol phosphate synthase enzyme (TrpC), a nucleic acid sequence encoding phosphoribosylanthranilate isomerase enzyme (TrpF), a nucleic acid sequence encoding α subunit of tryptophan synthase enzyme (TrpA) and/or a nucleic acid sequence encoding β subunit of tryptophan synthase enzyme (TrpB).

The present invention also relates to the use of a recombinant bacterium of the invention to produce anthranilic acid.

### BRIEF DESCRIPTION OF THE DRAWINGS

FIG. 1: Simplified representation of biosynthesis of anthranilic acid and aromatic amino acids.

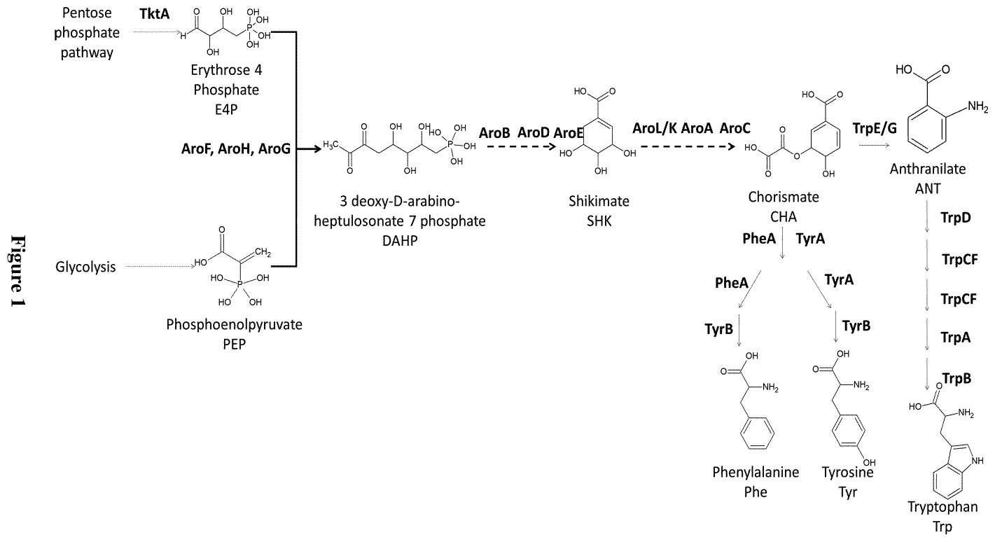

### DETAILED DESCRIPTION OF THE INVENTION

The present invention relates to a recombinant bacterium genetically modified in order to accumulate anthranilic acid while being able to grow in a culture medium lacking tryptophan. It also relates to the use of said recombinant bacterium to produce anthranilic acid and a method of producing anthranilic acid using said recombinant bacterium.

In the present application, the inventors showed that the carbon flux toward tryptophan pathway can be modified and controlled to limit the tryptophan production and to accumulate anthranilate. Indeed, they demonstrated that bacteria, in particular E. coli strains, can be genetically engineered in order to modulate the ratio between (i) anthranilate phosphoribosyltransferase activity and (ii) anthranilate synthase activity. By decreasing said ratio, the inventors showed that engineered strains are able to produce and accumulate significant amounts of anthranilic acid while being able to grow in a culture medium lacking tryptophan. Contrary to anthranilate producing bacteria described in the prior art, the recombinant bacteria of the invention do not exhibit tryptophan auxotrophy. These bacteria do not require any tryptophan supplementation and thus provide a great advantage over the strains of the prior art for the industrial production of anthranilic acid.

### Definitions

In the context of the invention, the term “recombinant bacterium” designates a bacterium that is not found in nature and which contains a modified genome as a result of either a deletion, insertion or modification of one or several genetic elements.

A “recombinant nucleic acid” or “recombinant nucleic acid molecule” designates a nucleic acid (such as, e.g., DNA, cDNA or RNA molecule) which has been engineered and is not found as such in nature or in wild-type bacteria. Typically, this term refers to a nucleic acid molecule comprising segments generated and/or joined together using recombinant DNA technology, such as for example molecular cloning and nucleic acid amplification. A recombinant nucleic acid molecule comprises one or more non-naturally occurring sequences, and/or contains joined nucleic acid molecules from different original sources and not naturally attached together.

The term “gene” designates any nucleic acid encoding a protein. This term encompasses DNA, such as cDNA or gDNA, as well as RNA. The gene may be first prepared by e.g., recombinant, enzymatic and/or chemical techniques, and subsequently replicated in a host cell or an in vitro system. The gene typically comprises an open reading frame encoding a desired protein. The gene may contain additional sequences such as a transcription terminator or a signal peptide.

As used herein, the term “operon” refers to a functioning unit of DNA containing two or more genes under the control of a single promoter.

As used herein, the term “expression cassette” denotes a nucleic acid construct comprising a coding region, i.e. one or several genes, and a regulatory region, i.e. comprising one or more control sequences including a transcriptional promoter, operably linked. Optionally, the expression cassette may comprise several coding regions operably linked to several regulatory regions. In particular, the expression cassette may comprise several coding sequences, each of these sequences being operably linked to the same promoter or to a distinct promoter. Alternatively, the expression cassette may comprise one or several coding sequences, each of these sequences operably linked to a distinct promoter, and several other coding sequences operably linked to a common promoter.

The term “operably linked” means a configuration in which a control sequence is placed at an appropriate position relative to a coding sequence, in such a way that the control sequence directs expression of the coding sequence.

The term “control sequences” means nucleic acid sequences necessary for expression of a gene. Control sequences may be native or heterologous. Well-known control sequences and currently used by the person skilled in the art will be preferred. Such control sequences include, but are not limited to, a leader, polyadenylation sequence, propeptide sequence, promoter, signal peptide sequence, ribosome binding site and transcription terminator. Preferably, the control sequences include a promoter and a transcription terminator.

As used herein, the term “expression vector” means a DNA or RNA molecule that comprises an expression cassette. Preferably, the expression vector is a linear or circular double stranded DNA molecule.

As used herein, the term “native” or “endogenous”, with respect to a host cell, refers to a genetic element or a protein naturally present in said host cell. The term “heterologous”, with respect to a host cell, refers to a genetic element or a protein that is not naturally present in said host cell. Preferably, this term refers to a genetic element or a protein provided from a cell of a different species or a different genus than the host cell.

As used herein, the term “sequence identity” or “identity” refers to the number (%) of matches (identical amino acid residues) in positions from an alignment of two polypeptide sequences. The sequence identity is determined by comparing the sequences when aligned so as to maximize overlap and identity while minimizing sequence gaps. In particular, sequence identity may be determined using any of a number of mathematical global or local alignment algorithms, depending on the length of the two sequences. Sequences of similar lengths are preferably aligned using a global alignment algorithms (e.g. Needleman and Wunsch algorithm; Needleman and Wunsch, 1970, J. Mol. Biol 48:443) which aligns the sequences optimally over the entire length, while sequences of substantially different lengths are preferably aligned using a local alignment algorithm (e.g. Smith and Waterman algorithm (Smith & Waterman, Adv. Appl. Math. 2:482, 1981) or Altschul algorithm (Altschul et al. 1997, Nucleic Acids Res. 25:3389-3402; Altschul et al. 2005, FEBS J. 272:5101-5109)). Alignment for purposes of determining percent amino acid sequence identity can be achieved in various ways that are within the skill in the art, for instance, using publicly available computer software available on internet web sites such as http://blast.ncbi.nlm.nih.gov/ or http://www.ebi.ac.uk/Tools/emboss/). Those skilled in the art can determine appropriate parameters for measuring alignment, including any algorithms needed to achieve maximal alignment over the full length of the sequences being compared. Preferably, for purposes herein, % amino acid sequence identity values refers to values generated using the pair wise sequence alignment program EMBOSS Needle that creates an optimal global alignment of two sequences using the Needleman-Wunsch algorithm, wherein all search parameters are set to default values, i.e. Scoring matrix=BLOSUM62, Gap open=10, Gap extend=0.5, End gap penalty=false, End gap open=10 and End gap extend=0.5. In some particular embodiments, all sequence identities mentioned in this application are identical and set to at least 70%, at least 75%, at least 80%, at least 85%, at least 90%, at least 95%, at least 96%, at least 97%, at least 98% or at least 99% sequence identity. In some more particular embodiments, all sequence identities mentioned in this application are identical and set to at least 80% sequence identity. In some other particular embodiments, all sequence identities mentioned in this application are identical and set to at least 90%, sequence identity.

The terms “peptide”, “oligopeptide”, “polypeptide” and “protein” are employed interchangeably and refer to a chain of amino acids linked by peptide bonds, regardless of the number of amino acids forming said chain.

As used herein, the term “activity” of an enzyme or an enzymatic complex refers to its function and designates in the context of the invention, the reaction that is catalysed by said enzyme or complex. The enzymatic activity may be measured by any method known by the skilled person.

The terms “overexpression” and “increased expression” as used herein, are used interchangeably and mean that the expression of a gene or an enzyme is increased compared to a non-modified bacterium. Increased expression of an enzyme is usually obtained by increasing expression of the gene encoding said enzyme. In embodiments wherein the gene or the enzyme is not naturally present in the bacterium of the invention, the terms “overexpression” and “expression” may be used interchangeably. To increase the expression of a gene, the skilled person can used any known techniques such as increasing the copy number of the gene in the bacterium, using a promoter inducing a high level of expression of the gene, i.e. a strong promoter, using elements stabilizing the corresponding messenger RNA or modifying Ribosome Binding Site (RBS) sequences and sequences surrounding them. In particular, the overexpression may be obtained by increasing the copy number of the gene in the bacterium. One or several copies of the gene may be introduced into the genome by methods of recombination, known to the expert in the field, including gene replacement or multicopy insertion. Preferably, an expression cassette comprising the gene, preferably placed under the control of a strong promoter, is integrated into the genome. Alternatively, the gene may be carried by an expression vector, preferably a plasmid, comprising an expression cassette with the gene of interest preferably placed under the control of a strong promoter. The expression vector may be present in the bacterium in one or several copies, depending on the nature of the origin of replication. The overexpression of the gene may also obtained by using a promoter inducing a high level of expression of the gene. For instance, the promoter of an endogenous gene may be replaced by a stronger promoter, i.e. a promoter inducing a higher level of expression. The promoters suitable to be used in the present invention are known by the skilled person and can be constitutive or inducible, preferably constitutive.

The term “decreased expression” as used herein, means that the expression of a gene or an enzyme is decreased compared to a non-modified bacterium. Decreased expression of an enzyme is usually obtained by decreasing expression of the gene encoding said enzyme. To decrease the expression of a gene, the skilled person can used any known techniques such as decreasing the copy number of the gene in the bacterium and/or using a promoter inducing a lower level of expression of the gene, i.e. a weak promoter Preferably, a decreased expression of a gene is obtained by using a promoter inducing a low level of expression of the gene. For instance, the promoter of an endogenous gene may be replaced by a weaker promoter, i.e. a promoter inducing a lower level of expression. The promoters suitable to be used in the present invention are known by the skilled person and can be constitutive or inducible, preferably constitutive.

As used herein, the term “non-modified bacterium” refers to the wild-type bacterium (i.e. naturally occurring bacterium) or the corresponding bacterium that has not been genetically modified in order to accumulate anthranilate, in particular the corresponding bacterium that has not been genetically modified to modulate the expression levels of genes encoding anthranilate phosphoribosyltransferase (TrpD), glutamine amidotransferase (TrpG) and/or anthranilate synthase (TrpE), and/or to modulate anthranilate phosphoribosyltransferase activity and/or anthranilate synthase activity. In particular, by comparison to a wild-type bacterium, the corresponding bacterium may comprise genetic modifications that are not directly linked to the production of anthranilate but provide advantages for the industrial production of anthranilate such as antibiotic resistance or enzymatic activities to enlarge substrate range. In preferred embodiments, this term refers to the wild-type bacterium (i.e. naturally occurring bacterium).

The term “anthranilate phosphoribosyltransferase” or “TrpD” refers to a polypeptide exhibiting anthranilate phosphoribosyltransferase activity (EC: 2.4.2.18), i.e. a polypeptide that catalyzes the transfer of a phosphoribosyl group of 5-phosphorylribose-1-pyrophosphate to anthranilate, forming N-(5′-phosphoribosyl)-anthranilate. The anthranilate phosphoribosyltransferase activity may be determined by any method known by the skilled person. For example, said activity may be assessed by fluorometric assay at 25° C. by measuring the rate of consumption of anthranilate (excitation wavelength, 310 nm; emission, 400 nm) in a spectrophotofluorimeter.

The term “glutamine amidotransferase” or “TrpG” refers to a polypeptide exhibiting glutamine amidotransferase activity, i.e. a polypeptide which generates ammonia as a substrate that, along with chorismate, is used in the second step, catalyzed by TrpE to produce anthranilate. The glutamine amidotransferase activity may be determined by any method known by the skilled person. For example, said activity may be assessed by monitoring p-nitroanilide formation from L-y-glutamyl-p-nitroanilide at 384 nm with a spectrophotometer.

The term “anthranilate synthase” or “TrpE” refers to a polypeptide exhibiting anthranilate synthase activity (EC:4.1.3.27), i.e. a polypeptide that catalyses the conversion of chorismate and glutamine to anthranilate, glutamate and pyruvate. In E. coli, this polypeptide is encoded by the trpE gene. As used herein, the term “anthranilate synthase activity” refers to the activity of converting chorismate and glutamine to anthranilate, glutamate and pyruvate, i.e. an activity resulting from the combination of TrpE and TrpG activities. The anthranilate synthase activity may be determined by any method known by the skilled person. For example, said activity may be assessed by monitoring the anthranilate formation from chorismate by following the increase in fluorescence (excitation wavelength, 310 nm; emission, 400 nm) in a spectrophotofluorimeter.

The term “indole-3-glycerol phosphate synthase” or “TrpC” refers to a polypeptide exhibiting indole-3-glycerol phosphate synthase activity (EC: 4.1.1.48). The term “phosphoribosylanthranilate isomerase” or “TrpF” refers to a polypeptide exhibiting phosphoribosylanthranilate isomerase activity (EC: 5.3.1.24). In a large variety of bacteria including E. coli, TrpC and TrpF are part of a bifunctional enzyme that catalyzes two sequential steps of tryptophan biosynthetic pathway. The first reaction is catalyzed by the isomerase, coded by the TrpF domain; the second reaction is catalyzed by the synthase, coded by the TrpC domain.

The term “α subunit of tryptophan synthase” or “TrpA” refers to an alpha-subunit of a tryptophan synthase, i.e. a polypeptide catalyzing the conversion of 1-C-(indol-3-yl)glycerol 3-phosphate to indole and D-glyceraldehyde 3-phosphate (EC: 4.2.1.20). The term “β subunit of tryptophan synthase” or “TrpB” refers to a beta-subunit of a tryptophan synthase. The indole formed by the alpha-subunit migrates to the beta-subunit where, in the presence of pyridoxal 5′-phosphate, it is combined with L-serine to form L-tryptophan.

According to the organism, the nomenclature of the above identified enzymes and encoding genes may vary. However, for the sake of clarity, in the present specification, these terms are used independently from the origin of the enzymes or genes.

As used herein, the term “tryptophan auxotroph” refers to a bacterium which is not able to synthesize tryptophan required for its growth. Such a bacterium cannot grow in a culture medium lacking tryptophan or any tryptophan source. On the contrary, the term “tryptophan non-auxotroph” refers to a bacterium which is able to synthesize tryptophan required for its growth. Such a bacterium can grow in a culture medium lacking tryptophan or any tryptophan source.

In the present application, the terms “anthranilic acid”, “o-aminobenzoic acid”, “2-aminobenzoic acid” and “anthranilate” can be used interchangeably and refer to an aromatic acid (CAS number: 118-92-3) having the formula (I) below

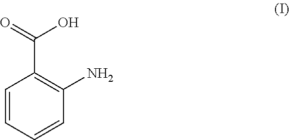

The inventors found that bacteria can be genetically engineered to control the carbon flux toward tryptophan pathway and accumulate anthranilate. Indeed, they demonstrated that bacteria genetically modified to induce an imbalance between anthranilate phosphoribosyltransferase activity and anthranilate synthase activity in favor of anthranilate synthase activity, are able to grow in a culture medium without tryptophan while being able to accumulate anthranilate.

Thus, in a first aspect, the present invention relates to a recombinant bacterium which is genetically modified to induce an imbalance between anthranilate phosphoribosyltransferase activity and anthranilate synthase activity in favor of anthranilate synthase activity. It also relates to a recombinant bacterium which is genetically modified to decrease the ratio between anthranilate phosphoribosyltransferase activity and anthranilate synthase activity, by comparison to the non-modified bacterium.

The recombinant bacterium of the invention still exhibits anthranilate phosphoribosyltransferase activity and is thus able to grow in a culture medium lacking tryptophan, i.e. is not tryptophan auxotroph. In other words, in the bacterium of the invention, the ratio between anthranilate phosphoribosyltransferase activity and anthranilate synthase activity is greater than zero. Indeed, TrpD activity remains sufficient to allow tryptophan synthesis and thus to allow growth of the bacterium in a culture medium lacking tryptophan or any tryptophan source.

Anthranilate is an intermediate in the tryptophan biosynthetic pathway and does not accumulate in wild-type bacteria which do not exhibit tryptophan auxotrophy. The recombinant bacterium of the invention comprises genetic modification(s) which is(are) designed to alter the carbon flux through the tryptophan pathway. These genetic modifications aim at inducing an imbalance between anthranilate synthase activity and TrpD activity in favor of anthranilate synthase activity. The recombinant bacterium thus produces more anthranilate than needed/used in the tryptophan biosynthesis, i.e. as substrate of TrpD. This imbalance leads to anthranilate accumulation and secretion while tryptophan is still produced by the bacterium thanks to TrpD remaining activity.

The anthranilate synthase activity is the result of a multimeric complex that catalyzes the two-step biosynthesis of anthranilate. In the first step, TrpG provides the glutamine amidotransferase activity which generates ammonia as a substrate that, along with chorismate, is used in the second step, catalyzed by TrpE to produce anthranilate. The anthranilate synthase activity is thus the result of TrpG and TrpE activities. In E. coli, anthranilate synthase is encoded by the trpE gene and the amino terminal region of the bifunctional protein encoded by the trpGD gene is responsible of glutamine amidotransferase (TrpG) activity. Indeed, in a large variety of bacteria, including but not limited to E. coli, a bifunctional protein is responsible of TrpG and TrpD activities. For example, in E. coli, the amino terminal region of the bifunctional protein encoded by the trpGD gene is responsible of TrpG activity while the carboxy terminal region of this protein is responsible of TrpD activity. With the present invention, the inventors found that expression of the TrpG domain of this bifunctional protein can be decoupled from expression of the TrpD domain allowing fine-tuning of the balance between anthranilate phosphoribosyltransferase activity and anthranilate synthase activity.

In the recombinant bacterium of the invention, the imbalance between anthranilate phosphoribosyltransferase activity and anthranilate synthase activity may be obtained in various different ways but always in favor of anthranilate synthase activity. Despite of this imbalance, the recombinant bacterium still exhibits a sufficient level of anthranilate phosphoribosyltransferase activity to synthetize tryptophan required for its growth. In some embodiments, TrpE and/or TrpG activities are increased. In these embodiments, TrpD activity may remain unaltered, may be decreased or may be increased but to a lesser extent than anthranilate synthase activity resulting from TrpE and TrpG activities. Preferably, in these embodiments, TrpD activity remains unaltered or is decreased. In some preferred embodiments, TrpD activity is decreased. In these embodiments, anthranilate synthase activity resulting from TrpE and TrpG activities may remain unaltered, may be increased or may be decreased but to a lesser extent than TrpD activity. Preferably, in these embodiments, anthranilate synthase activity resulting from TrpE and TrpG activities remains unaltered or is increased.

As used herein, the term “increased activity” refers to an enzymatic activity that is increased in the recombinant bacterium of the invention by comparison to the non-modified bacterium. This increase may be due to increased specific catalytic activity, increased specificity for the substrate, increased protein or RNA stability and/or increased intracellular concentration of the enzyme or enzymatic complex responsible of this activity. Preferably, the increased activity is due to an increased intracellular concentration of the enzyme obtained by overexpressing a gene encoding said enzyme (overexpression of a endogenous gene or expression of a heterologous gene). When the enzymatic activity results from the activity of two distinct polypeptides (e.g. TrpE and TrpG for anthranilate synthase activity), the increased activity may be due to an increased intracellular concentration of one or both, preferably both, of these polypeptides obtained by overexpressing the gene(s) encoding said polypeptide(s).

As used herein, the term “decreased activity” refers to an enzymatic activity that is decreased in the recombinant bacterium of the invention by comparison to a non-modified bacterium. This decrease may be due to decreased specific catalytic activity, decreased specificity for the substrate, decreased protein or RNA stability and/or decreased intracellular concentration of the enzyme or enzymatic complex responsible of this activity. In some embodiments, the decreased activity may be due to a decreased intracellular concentration of the enzyme. The decreased intracellular concentration of the enzyme may be obtained by decreasing the level of expression of the gene encoding said enzyme or by changes affecting RNA stability and thus translation efficiency. When the enzymatic activity results from the activity of two distinct polypeptides (e.g. TrpE and TrpG for anthranilate synthase activity), the decreased activity may be due to a decreased intracellular concentration of one or both of these polypeptides. In some other embodiments, the decreased activity may be due to a decreased specific activity of the enzyme. In particular, the specific activity may be decreased due to structural changes, e.g. when a domain of a bifunctional protein is expressed separately from the other domain. As used herein, this term excludes the total suppression of said enzymatic activity.

As mentioned above, the inventors found that decoupling expression of TrpG polypeptide from expression of TrpD polypeptide allows fine-tuning of the balance between anthranilate phosphoribosyltransferase activity and anthranilate synthase activity. Thus, in a more particular aspect, the present invention relates to a recombinant bacterium which has been genetically modified to decouple expression of TrpG polypeptide from expression of TrpD polypeptide.

The recombinant bacterium comprises a nucleic acid sequence encoding glutamine amidotransferase (TrpG) and a nucleic acid sequence encoding anthranilate phosphoribosyltransferase (TrpD) leading to the expression of two distinct polypeptides, i.e. a polypeptide exhibiting glutamine amidotransferase activity (TrpG; does not exhibit anthranilate phosphoribosyltransferase activity) and a polypeptide exhibiting anthranilate phosphoribosyltransferase activity (TrpD; does not exhibit glutamine amidotransferase activity). Expression of said nucleic acid sequences encoding glutamine amidotransferase (TrpG) and anthranilate phosphoribosyltransferase (TrpD) may be placed under the control of the same promoter or under the control of two different promoters. Preferably, the recombinant bacterium comprises/expresses a nucleic acid sequence encoding glutamine amidotransferase (TrpG) under the control of a first promoter and a nucleic acid sequence encoding anthranilate phosphoribosyltransferase (TrpD) under the control of a second promoter.

As used herein, the term “nucleic acid sequence encoding TrpG”, “nucleic acid sequence encoding glutamine amidotransferase” or “a gene encoding TrpG” refers to a nucleic acid encoding a polypeptide that exhibits glutamine amidotransferase activity and does not exhibit anthranilate phosphoribosyltransferase activity, e.g. a polypeptide corresponding only to a TrpG domain of a bifunctional protein TrpGD. Similarly, the term “nucleic acid sequence encoding TrpD”, “nucleic acid sequence encoding anthranilate phosphoribosyltransferase” or “a gene encoding TrpD” refers to a nucleic acid encoding a polypeptide that exhibits anthranilate phosphoribosyltransferase activity and does not exhibit glutamine amidotransferase activity, e.g. a polypeptide corresponding only to a TrpD domain of a bifunctional protein TrpGD.

Preferably, in bacteria wherein, in their wild-type form, endogenous TrpG and TrpD activities are expressed as a bifunctional protein TrpGD, the recombinant bacterium of the invention is genetically modified to suppress expression of said endogenous bifunctional protein TrpGD. The endogenous TrpGD gene may be inactivated by any method known by the skilled person, for example by deletion of all or part of this gene, by introducing a nonsense codon or a mutation inducing a frameshift, or by insertion of a gene or an expression cassette. In a particular embodiment, expression of only one of the domains, i.e. TrpD or TrpG, of the endogenous bifunctional protein TrpGD is suppressed, more preferably expression of only TrpD domain is suppressed. Expression of TrpD domain can be suppressed by any method known by the skilled person, e.g. by deleting all or part of the nucleic acid sequence encoding said domain, by introducing a nonsense codon or a mutation inducing a frameshift, or by insertion of a gene or an expression cassette. Preferably, expression of TrpD domain is suppressed by deleting all or part of the nucleic acid sequence encoding said domain. In some embodiments, in particular when the bacterium is Escherichia coli, expression of TrpD domain is suppressed by deleting part of the nucleic acid sequence encoding said domain while preserving the activity of the internal promoter TrpCp, an internal low efficiency promoter which is not repressed by tryptophan (Horowitz and Platt, Journal of Molecular Biology, 156.2 (1982), 257-67).

In embodiments wherein the endogenous nucleic acid sequence encoding TrpD activity is inactivated, the recombinant bacterium may be further modified by introducing an expression cassette comprising an endogenous or heterologous gene encoding TrpD. In said embodiments, expression of the endogenous TrpG polypeptide is preferably maintained.

In embodiments wherein endogenous gene(s) encoding TrpD and TrpG activities is(are) inactivated, the recombinant bacterium may be further modified by introducing one or several expression cassettes comprising an endogenous or heterologous gene encoding TrpG and an endogenous or heterologous gene encoding TrpD, said genes leading to the expression of two distinct polypeptides. In particular, the recombinant bacterium may be further modified by introducing an expression cassette comprising an endogenous or heterologous gene encoding TrpG and an expression cassette comprising an endogenous or heterologous gene encoding TrpD.

In bacteria wherein TrpG and TrpD activities are naturally expressed as a bifunctional protein TrpGD, the terms “endogenous gene encoding TrpG” and “endogenous gene encoding TrpD” refers to the endogenous nucleic acid sequence encoding domain TrpG of the bifunctional protein and the endogenous nucleic acid sequence encoding domain TrpD of the bifunctional protein, respectively. TrpD domain or polypeptide does not exhibit glutamine amidotransferase activity and TrpG domain or polypeptide does not exhibit anthranilate phosphoribosyltransferase activity.

As mentioned above, TrpG and TrpD polypeptides expressed in the recombinant bacterium of the invention may be endogenous or heterologous polypeptides (the both may be endogenous or heterologous, or one may be endogenous and the other heterologous). In particular, TrpG and TrpD polypeptides may be any known TrpG and TrpD polypeptides, preferably selected from bacterial TrpG and TrpD polypeptides. In particular, TrpG and TrpD polypeptides may be selected from TrpG and TrpD domains of bacterial bifunctional proteins TrpGD.

The nucleic acid sequence encoding TrpG may be selected from the group consisting of nucleic acid sequences encoding the TrpG domain of TrpGD protein of Escherichia coli (from position 3 to position 196 of SEQ ID NO: 1), TrpG of Bacillus subtilis (Uniprot accession number: P28819; SEQ ID NO: 2), the TrpG domain of TrpGD protein of Salmonella typhimurium (Uniprot accession number: P00905; from position 3 to position 196 of SEQ ID NO: 3), TrpG of Cupriavidus necator (Uniprot accession number: QOK6I2; SEQ ID NO: 4) and TrpG of Corynebacterium glutamicum (Uniprot accession number: P06558; SEQ ID NO: 5). The TrpG polypeptide may also be any polypeptide exhibiting TrpG activity and having at least 70%, preferably at least 75%, at least 80%, at least 85%, at least 90%, at least 95%, at least 96%, at least 97%, at least 98% or at least 99%, identity to any of SEQ ID NO: 2, 4 and 5, sequence from position 3 to position 196 of SEQ ID NO: 1 and sequence from position 3 to position 196 of SEQ ID NO: 3.

The nucleic acid sequence encoding TrpD may be selected from the group consisting of nucleic acid sequences encoding the TrpD domain of TrpGD protein of Escherichia coli (strain K12) (from position 202 to position 531 of SEQ ID NO: 1), TrpD of Bacillus subtilis (Uniprot accession number: P03947; SEQ ID NO: 6), the TrpD domain of TrpGD protein of Salmonella typhimurium (Uniprot accession number: P00905; from position 202 to position 531 of SEQ ID NO: 3), TrpD of Cupriavidus necator (Uniprot accession number: QOK6I1; SEQ ID NO: 7) and TrpD of Corynebacterium glutamicum (Uniprot accession number: P06559; SEQ ID NO: 8). The TrpD polypeptide may also be any polypeptide exhibiting TrpD activity and having at least 70%, preferably at least 75%, at least 80%, at least 85%, at least 90%, at least 95%, at least 96%, at least 97%, at least 98% or at least 99%, identity to any of SEQ ID NO: 6, 7 and 8, sequence from position 202 to position 531 of SEQ ID NO: 1 and sequence from position 202 to position 531 of SEQ ID NO: 3.

Other TrpD and TrpG polypeptides may be easily identified using routine methods, for example based on homology with polypeptides listed above.

Preferably, TrpD and TrpG polypeptides originate from the same bacterial species. More preferably, TrpD and TrpG polypeptides originate from the same species than the recombinant bacterium of the invention.

The recombinant bacterium of the invention has been genetically modified to induce an imbalance between anthranilate phosphoribosyltransferase activity and anthranilate synthase activity in favor of anthranilate synthase activity.

In some embodiments, this imbalance is induced by adjusting the expression level of TrpD and the expression level of TrpG and/or TrpE, preferably the expression level of TrpG, more preferably the expression level of TrpG and TrpE. Indeed, in these embodiments, the expression level of TrpD is lower than the expression level of TrpG and/or TrpE, preferably lower than the expression level of TrpG, more preferably lower than the expression level of TrpG and TrpE. In particular, the expression level of TrpD may be at least 10%, at least 20%, at least 30%, at least 40%, at least 50%, at least 60%, at least 70%, at least 80%, or at least 90% lower than the expression level of TrpG or TrpE, preferably at least 10%, at least 20%, at least 30%, at least 40%, at least 50%, at least 60%, at least 70%, at least 80%, or at least 90% lower than the expression level of TrpG, more preferably at least 10%, at least 20%, at least 30%, at least 40%, at least 50%, at least 60%, at least 70%, at least 80%, or at least 90% lower than the expression level of TrpG and at least 10%, at least 20%, at least 30%, at least 40%, at least 50%, at least 60%, at least 70%, at least 80%, or at least 90% lower than the expression level of TrpE.

The expression level of a polypeptide can be determined by a variety of techniques. In particular, the expression level may be determined by measuring the quantity of said polypeptide or the corresponding mRNA. Preferably, the expression level is determined by measuring the quantity of the corresponding mRNA. Methods for determining the quantity of mRNA are well known in the art. For example, the nucleic acid contained in the bacterium is first extracted according to standard methods, for example using lytic enzymes or chemical solutions or extracted by nucleic-acid-binding resins following the manufacturer's instructions. The extracted mRNA is then detected by hybridization (e. g., Northern blot analysis) and/or amplification (e.g., RT-PCR). Preferably, mRNA corresponding to the polypeptide is detected and quantified by quantitative or semi-quantitative RT-PCR. Real-time quantitative or semi-quantitative RT-PCR is particularly advantageous. Primer pairs are designed in order to specifically detected and quantified polypeptide(s) of interest.

The recombinant bacterium of the invention may comprise a gene encoding TrpG under the control of a first promoter and a gene encoding TrpD under the control of a second promoter. The first and the second promoters may be two different promoters exhibiting different strength/rate of transcription. Preferably, the first promoter is a promoter that is not operably linked to a gene encoding TrpG in the non-modified bacterium and/or the second promoter is a promoter that is not operably linked to a gene encoding TrpD in the non-modified bacterium.

In a particular embodiment, the recombinant bacterium of the invention comprises
a gene encoding TrpG or a first operon under the control of a first promoter, wherein said first operon comprises at least a nucleic acid sequence encoding TrpG and optionally a nucleic acid sequence encoding TrpE, and
a gene encoding TrpD or a second operon under the control of a second promoter, wherein said second operon comprises at least a nucleic acid sequence encoding TrpD.

In said embodiment, the recombinant bacterium of the invention comprises a recombinant nucleic acid comprising the gene encoding TrpG or the first operon under the control of the first promoter and/or a recombinant nucleic acid comprising the gene encoding TrpD or the second operon under the control of the second promoter. Preferably, the first promoter is a promoter that is not operably linked to a gene encoding TrpG in the non-modified bacterium and/or the second promoter is a promoter that is not operably linked to a gene encoding TrpD in the non-modified bacterium.

The first or second operon, preferably the second operon, may further comprise a nucleic acid sequence encoding indole-3-glycerol phosphate synthase (TrpC) and/or a nucleic acid sequence encoding phosphoribosylanthranilate isomerase (TrpF), and/or a nucleic acid sequence encoding α subunit of tryptophan synthase (TrpA), and/or a nucleic acid sequence encoding β subunit of tryptophan synthase (TrpB).

The genes encoding TrpG and TrpD or the first and second operons may be comprised in one or several recombinant nucleic acids comprising one or several expression cassettes. Preferably, expression cassettes used in the present invention comprise at least a promoter operably linked to a gene or an operon and a transcription terminator which is recognized by the bacterium to terminate transcription. The terminator is operably linked to the 3′-terminus of the encoding nucleic acid. Any terminator that is functional in the host cell may be used in the present invention and can be easily chosen by the skilled person. Usually, the terminator is chosen in correlation with the promoter.

In an embodiment, the recombinant bacterium of the invention comprises a first recombinant nucleic acid comprising a first expression cassette comprising a gene encoding TrpG or the first operon as defined above, and a second recombinant nucleic acid comprising a second expression cassette comprising a gene encoding TrpD or the second operon as defined above.

In another embodiment, the recombinant bacterium of the invention comprises a recombinant nucleic acid comprising a first expression cassette comprising a gene encoding TrpG or the first operon as defined above and a second expression cassette comprising a gene encoding TrpD or the second operon as defined above.

The first operon comprising a nucleic acid sequence encoding TrpG and optionally a nucleic acid sequence encoding TrpE, may be a chromosomal endogenous operon having been modified to inactivate or to not express TrpD. In this case, the promoter controlling expression of said operon may be the promoter controlling expression of TrpG, and optionally TrpE, in the non-modified bacterium.

In some embodiments, and in particular when the non-modified bacterium expresses two distinct polypeptides, one exhibiting TrpD activity and the other expressing TrpG activity, the expression level of TrpG and/or TrpE, preferably TrpG and TrpE, in the recombinant bacterium of the invention, is higher than the expression level of TrpD. Optionally, the expression level of TrpG and/or TrpE, preferably TrpG and TrpE, may also be higher than expression level of TrpC, TrpF, TrpA and/or TrpB, preferably TrpD, TrpF, TrpA, TrpC and TrpB.

In some other embodiments, and in particular when the non-modified bacterium expresses a bifunctional protein exhibiting TrpD and TrpG activities, the expression level of TrpG and/or TrpE, preferably TrpG and TrpE, in the recombinant bacterium of the invention, may be higher, lower or substantially equal, preferably higher or substantially equal, to the expression level of TrpD. Indeed, the inventors showed that a recombinant bacterium genetically modified to separately express a polypeptide exhibiting glutamine amidotransferase activity (TrpG) (and not exhibiting anthranilate phosphoribosyltransferase activity) and a polypeptide exhibiting anthranilate phosphoribosyltransferase activity (TrpD) (and not exhibiting glutamine amidotransferase activity), accumulates anthranilic acid even if expression of TrpD is controlled by a strong promoter.

The expression level of a gene or operon may be altered by any method known by the skilled person, in particular by adjusting the copy number of the gene or operon and/or adjusting the strength of the promoter controlling its expression.

The promoters used in the present invention may be any polynucleotide that shows transcriptional activity in the host cell including mutant, truncated, and hybrid promoters, and may be obtained from endogenous or heterologous genes. The promoters used to control the expression of a gene encoding TrpD or an operon comprising said gene and the expression of a gene encoding TrpG or an operon comprising said gene may be constitutive or inducible. In particular, the activity of the promoter controlling the expression of a gene encoding TrpD or an operon comprising said gene, may be regulated by tryptophan concentration.

In preferred embodiments, the promoter controlling expression of a gene encoding TrpG or an operon comprising said gene is stronger than the promoter controlling expression of a gene encoding TrpD or an operon comprising said gene. In particular, the promoter controlling expression of a gene encoding TrpG or an operon comprising said gene, may be a strong promoter, i.e. a promoter leading to a high rate of transcription initiation, while the promoter controlling expression of a gene encoding TrpD or an operon comprising said gene, may be a weak promoter, i.e. a promoter leading to a low rate of transcription initiation. Alternatively, both promoters may be strong promoters or weak promoters provided that the imbalance between anthranilate phosphoribosyltransferase activity and anthranilate synthase activity remains in favor of anthranilate synthase activity. Examples of such strong promoters include, but are not limited to, T7 promoter, pTac, pRecA and pTrp. Examples of such weak promoters include, but are not limited to, pLac and pLacUV5. Further promoters are described in Sambrook et al., 2012; Molecular cloning: a laboratory manual, fourth, Edition Cold Spring Harbor. In a particular embodiment, the promoter controlling expression of a gene encoding TrpG or an operon comprising said gene is pTrp or a promoter of same strength, preferably pTrp, and the promoter controlling expression of a gene encoding TrpD or an operon comprising said gene is pTac or a promoter of same strength, preferably pTac.

In a more particular embodiment, the wild-type bacterium corresponding to the recombinant bacterium, preferably E. coli, expresses a bifunctional protein exhibiting TrpG and TrpD activities and the recombinant bacterium has been genetically modified to separately express a polypeptide exhibiting glutamine amidotransferase activity (TrpG) (and not exhibiting anthranilate phosphoribosyltransferase activity) under the control of a first promoter and a polypeptide exhibiting anthranilate phosphoribosyltransferase activity (TrpD) (and not exhibiting glutamine amidotransferase activity) under the control of a second promoter, both promoters being strong promoters. Preferably, the promoter controlling expression of a gene encoding TrpG or an operon comprising said gene is pTrp or a promoter of same strength, preferably pTrp, and the promoter controlling expression of a gene encoding TrpD or an operon comprising said gene is pTac or a promoter of same strength, preferably pTac.

Optionally, the recombinant nucleic acid(s) used in the invention may also comprise a selectable marker that permits easy selection of recombinant host cells. Typically, the selectable marker is a gene encoding antibiotic resistance.

Expression vector(s) comprising the recombinant nucleic acid(s) as disclosed above, may be used to transform the bacterium. The choice of the expression vector typically depends on the compatibility of the vector with the host cell into which the vector is to be introduced. The vector may be an autonomously replicating vector, i.e., a vector that exists as an extra-chromosomal entity, the replication of which is independent of chromosomal replication, e.g., a plasmid, an extra-chromosomal element, a mini-chromosome, or an artificial chromosome. The vector may contain any means for assuring self-replication. Alternatively, the vector may be one that, when introduced into the host cell, is integrated into the genome and replicated together with the chromosome(s) into which it has been integrated. Preferably, the vector, or a part thereof comprising at least one recombinant nucleic acid disclosed herein, is inserted in the genome of the bacterium. When integration into the host cell genome occurs, integration of the sequences into the genome may rely on homologous or non-homologous recombination. In one hand, the vector may contain additional polynucleotides for directing integration by homologous recombination at a precise location into the genome of the host cell. These additional polynucleotides may be any sequence that is homologous with the target sequence in the genome of the host cell. On the other hand, the vector may be integrated into the genome of the host cell by non-homologous recombination. For autonomous replication, the vector may further comprise an origin of replication enabling the vector to replicate autonomously in the host cell in question. The origin of replication may be any plasmid replicator mediating autonomous replication that functions in a cell. The vector preferably also comprises one or more selectable markers that permit easy selection of host cells comprising the vector. Typically, the selectable marker is a gene encoding antibiotic resistance. The methods for selecting these elements according to the host cell are well known to one of skill in the art. The expression vectors may be constructed by the classical techniques of molecular biology, well known to one of skill in the art. The recombinant nucleic acid(s) comprised in the recombinant bacterium of the invention may be integrated into the genome of the bacterium or may be maintained in an episomal form into one or several expression vectors. In embodiments wherein the recombinant nucleic acid(s) maintained in an episomal form, the expression vector(s) may be present in the bacterium in one or several copies, depending on the nature of the origin of replication. Preferably, the recombinant nucleic acid(s) is(are) integrated into the genome of the bacterium. One or several copies of the recombinant nucleic acid(s) may be introduced into the genome by methods of recombination, known to the expert in the field, including gene replacement. Preferably, in embodiments wherein the second recombinant nucleic acid is maintained in an episomal form into an expression vector, said expression vector is present in the bacterium in less than 25 copies, more preferably less than 10 copies.

In a particular embodiment, the recombinant bacterium of the invention comprises a first recombinant nucleic acid comprising a first expression cassette comprising a gene encoding TrpG or the first operon as defined above, said first recombinant nucleic acid being integrated into the genome, and a second recombinant nucleic acid comprising a second expression cassette comprising a gene encoding TrpD or the second operon as defined above, said second recombinant nucleic acid being maintained in an episomal form into an expression vector.

The recombinant bacterium may be further genetically modified to increase carbon flux towards anthranilic acid.

In particular, the recombinant bacterium of the invention may be genetically modified to express a heterologous gene encoding a feedback resistant 3-deoxy-D-arabino-heptulosonate-7-phosphate synthase enzyme. The enzyme 3-deoxy-D-arabino-heptulosonate 7-phosphate synthase (DAHP synthase, EC 4.1.2.15) catalyzes the condensation of phospho(enol)pyruvate (PEP) and erythrose-4-phosphate (E4P) to DAHP and inorganic phosphate. This reaction is the first committed step in the biosynthesis of aromatic compounds in microorganisms. Escherichia coli has three DAHP synthase isoenzymes, each of which is feedback-regulated by one of the aromatic amino acids, tyrosine, phenylalanine, and tryptophan. As used herein, the term “feedback resistant 3-deoxy-D-arabino-heptulosonate-7-phosphate synthase” or “feedback resistant DAHP synthase” refers to a DAHP synthase which is not negatively regulated by a product of the shikimate pathway, i.e. a tyrosine-, phenylalanine- and/or tryptophan-insensitive DAHP synthase. Feedback resistant 3-deoxy-D-arabino-heptulosonate-7-phosphate synthase may be any feedback resistant DAHP synthase known by the skilled person, preferably a bacterial feedback resistant DAHP synthase. Examples of feedback resistant DAHP synthases include, but are not limited to, E. coli AroF with Asn-8 to Lys-8 substitution (Jossek et al. FEMS Microbiol Lett, 2001 Aug. 7; 202(1):145-8), AroG with Ala-146 to Ans-146 substitution (Mascarenhas et al. Applied and Environmental Microbiology, 1991 oct, 57; 10:2995-2999), AroH with Pro-18 to Lys-18 substitution, Val-147 to Met-147 substitution, Gly-149 to Asp-149 substitution, Gly-149 to Cys-149 substitution or Ala-177 to Tyr-177 substitution (Ray et al. Journal of Bacteriology, 1988 December 170(12) 5500-5506).

Alternatively, or in addition, the recombinant bacterium of the invention may be further genetically modified to overexpress an endogenous gene encoding a transketolase enzyme or express a heterologous gene encoding a transketolase enzyme. Transketolase (EC 2.2.1.1) catalyzes the reversible transfer of a ketol group between several donor and acceptor substrates. This enzyme is a reversible link between glycolysis and the pentose phosphate pathway. The enzyme is involved in the catabolism of pentose sugars and the provision of erythrose-4-phosphate (E4P), a precursor of aromatic amino acids. E. coli contains two transketolase isozymes, TktA and TktB. Preferably, the recombinant bacterium of the invention is genetically modified to overexpress a endogenous gene encoding a transketolase enzyme.

The recombinant nucleic acid(s) or expression vector(s) disclosed above may be introduced into the bacterium by any method known by the skilled person, such as electroporation, conjugation, transduction, competent cell transformation, protoplast transformation or protoplast fusion. According to the nature of the host cell, the skilled person can easily chose a suitable method.

The recombinant bacterium of the invention is preferably obtained through genetic modification of a bacterium having the metabolic capacity to synthesize tryptophan. In particular, the recombinant bacterium of the invention is preferably obtained from a bacterium having the metabolic capacity to synthesize tryptophan and wherein endogenous TrpG and TrpD activities in the non-modified bacterium are expressed as a bifunctional protein TrpGD.

The recombinant bacterium may be any Gram-positive or Gram-negative bacterium. Examples of suitable bacteria include, but are not limited to, bacteria of the genus Escherichia (e.g. Escherichia coli), Streptomyces, Bacillus, Cupridavidus, Corynebacterium Mycobacterium, Kitasatospora, Luteipulveratus, Thermobifida, Thermomonospora, Frankia, Pseudonocardia, Saccharothrix, Kutzneria, Lentzea, Prauserella, Salinispora, Micromonospora, Actinoplanes, Catenulispora, Mycolicibacterium, Dietzia, Aeromicrobium, Nonomuraea, Blastococcus, Modestobacter, Saccharopolyspora, Amycolatopsis, Actinopolyspora, Acidimicrobium, Photorhabdus, Hoeflea, Azospirillum, Crinalium or Cylindrospermum. Preferably, the recombinant bacterium of the invention is selected from bacteria of the genera Escherichia, Streptomyces, Corynebacterium and Bacillus. More preferably, the recombinant bacterium of the invention is selected from bacteria of the genera Escherichia, Streptomyces and Bacillus. Even More preferably, the recombinant bacterium of the invention is Escherichia coli.

In a particular embodiment, the recombinant bacterium is an Escherichia coli bacterium which has been genetically modified to comprise a nucleic acid sequence encoding glutamine amidotransferase (TrpG) under the control of a first promoter and a nucleic acid sequence encoding anthranilate phosphoribosyltransferase (TrpD) under the control of a second promoter. Preferably, said recombinant bacterium comprises (i) a gene encoding glutamine amidotransferase (TrpG) or a first operon, under the control of a first promoter, wherein said first operon comprises at least a nucleic acid sequence encoding TrpG and optionally a nucleic acid sequence encoding anthranilate synthase (TrpE), and (ii) a gene encoding anthranilate phosphoribosyltransferase (TrpD) or a second operon, under the control of a second promoter wherein said second operon comprises at least a nucleic acid sequence encoding TrpD, and optionally a nucleic acid sequence encoding indole-3-glycerol phosphate synthase enzyme (TrpC), a nucleic acid sequence encoding phosphoribosylanthranilate isomerase enzyme (TrpF), a nucleic acid sequence encoding α subunit of tryptophan synthase enzyme (TrpA) and/or a nucleic acid sequence encoding β subunit of tryptophan synthase enzyme (TrpB). Both promoters may be strong promoters. Preferably, the promoter controlling expression of a gene encoding TrpG or an operon comprising said gene is pTrp or a promoter of same strength, preferably pTrp, and the promoter controlling expression of a gene encoding TrpD or an operon comprising said gene is pTac or a promoter of same strength, preferably pTac.

Preferably, the recombinant bacterium of the invention does not exhibit salicylate 5-hydroxylase (S5H) activity and/or does not produce 5-hydroxyanthranilate (5-HAA).

Preferably, the recombinant bacterium of the invention is able to produce at least 1 g/L of anthranilate when cultured in 2 L fed-batch fermenter or at least 4 g/L of anthranilate when cultured in 20 L fed-batch fermenter, during 48 h in the presence of glucose as carbon source. Alternatively, the recombinant bacterium of the invention may be able to produce at least 300 mg/L, preferably at least 400 mg/L, more preferably at least 500 mg/L, when cultured in 5 mL-culture during 30 h in the presence of glucose as carbon source.

In a further aspect, the present invention relates to the use of a recombinant bacterium of the invention, to produce anthranilic acid. The present invention also relates to a method of producing anthranilic acid, comprising culturing a recombinant bacterium of the invention under conditions suitable to produce said compound, and optionally recovering said anthranilic acid. Anthranilic acid is secreted by bacteria. Thus, anthranilic acid may be recovered by collecting the culture medium and in particular the culture supernatant. The method may further comprise isolating or purifying said anthranilic acid. anthranilic acid may be isolated or purified using any method known by the skilled person such as precipitation, ion exchange, reverse osmosis and filtration.

Preferably, the recombinant bacterium used in the method of the invention does not produce 5-hydroxyanthranilate (5-HAA) and anthranilic acid produced and optionally recovered, is deprived of 5-HAA.

All embodiments described above for the recombinant bacterium of the invention are also contemplated in these aspects.

Conditions suitable to produce anthranilic acid may be easily determined by the skilled person according to the recombinant bacterium used. In particular, the skilled person may easily chose suitable culture medium and growth conditions according to the bacterium. Preferably, the culture medium used to produce anthranilic acid with a recombinant bacterium of the invention is deprived of tryptophan or any tryptophan source or is not supplemented with tryptophan.

Further aspects and advantages of the present invention will be described in the following examples, which should be regarded as illustrative and not limiting.

### EXAMPLES

### Example 1

Material and Methods

Strains Construction

Strain K1 was constructed by replacing anthranilate phosphoribosyl transferase domain of trpD of MG1655 E. coli strain by a chloramphenicol cassette using a lambda red method (Kirill et al. PNAS, June 2000, 97 (12) 6640-6645). Briefly, primers 1 and 2 (see table 1 below) comprising sequence homologies with trpD gene were used to amplify chloramphenicol cassette of pKD3 plasmid (Kirill et al. PNAS, June 2000, 97 (12) 6640-6645). MG1655 cells were then transformed with pKD46 plasmid (Kirill et al. PNAS, June 2000, 97 (12) 6640-6645) and with this PCR product thereby allowing lambda red recombination.

Strain K2 was constructed by removing the chloramphenicol cassette of K1 strain using pCP20 plasmid (Kirill et al. PNAS, June 2000, 97 (12) 6640-6645). Strain K2 was then transformed by p002 plasmid (see below), leading to K3 strain.

Plasmids Construction

The low copy plasmid pBAC-LacZ (addgene no 13422) was used as backbone. lacZ gene was removed by a Sal digestion of the pBAC-LacZ plasmid leading to pBAC-Δ(LacZ) plasmid. Then, pTAC promoter (a hybrid of the E. coli trp and lac promoters) and rrnB T1 terminator (transcription terminator T1 from the E. coli rrnB gene) were added to this plasmid by a digestion/ligation method (SalI/HpaI and SalI/ZraI). The anthranilate phosphoribosyl transferase domain of trpD was amplified from E. coli chromosome with primers 3 and 4 (see table 2 below) and then cloned into pBAC-Δ(LacZ) between the pTAC promoter and rrnB T1 terminator by a digestion/ligation (NheI/HindIII), leading to plasmid p002.

TABLE 2
List of primers

| Primer | |
|---|---|
| name | sequence |
| 1 | gcgcagcagaaactagagccagccaacacgct |
| | gcaaccgattctgtaagtgtaggctggagctg |
| | cttc (SEQ ID NO: 9) |
| 2 | aatcgccttgtctgcgacgattttcgctaaaa |
| | cggtttgcatcatatgggaattagccatggtc |
| | c (SEQ ID NO: 10) |
| 3 | agtatggctagcttaccctcgtgccgccagtg |
| | (SEQ ID NO: 11) |
| 4 | gactagaagcttcctctagaaataattttgtt |
| | taactttaagaaggagaatcgatatgaacacg |
| | ctgcaaccgattctg (SEQ ID NO: 12) |

Culture Conditions

K2 and K3 strains were cultured in 5 ml of LB medium during 8 h, then overnight in 10 ml of mineral medium M9 (the composition of the medium is described in table 3). 20 mg/l of tryptophan were added in K2 culture. 100 μl of each preculture were used to inoculate 25 ml of mineral medium M9 in the same conditions as preculture. The strains were then cultured during 31 h at 37° C.

TABLE 3
Composition of M9 culture medium

| | g/l |
|---|---|
| Na₂HPO₄ | 6.9 |
| KH₂PO₄ | 3.03 |
| NaCl | 0.51 |
| NH₄Cl | 2.04 |
| MgSO₄, 7H₂O | 0.49 |
| CaCl₂, 2H₂O | 0.00438 |
| Na₂MoO₄, 2H₂O | 0.015 |
| ZnSO₄, 7H₂O | 0.0045 |
| CoCl₂, 6H₂O | 0.0003 |
| MnCl₂, 4H₂O | 0.001 |
| H₃BO₃ | 0.001 |
| Na₂MoO₄, 2H₂O | 0.0004 |
| FeSO₄, 7H₂O | 0.003 |
| CuSO₄, 5H₂O | 0.0003 |
| Thiamine HCl | 0.1 |

Analysis

The quantity of anthranilate in the supernatant of the cultures was measured using an LC-UV method. The HPLC instrument was equipped with an column (Waters Acquity BEH C18 (50*2.1 mm; 1.7 μm)) and coupled to a UV detector: [190-400 nm]. Solution of H2O/ACN was used as a mobile phase at 40° C. at flow rate of 0.5 ml/, a linear elution gradient from 5% to 100% ACN over 8 min and an UV detector (O210 nm).

Results

Strain K2 cultivated in M9 medium without tryptophan did not grown nor produced anthranilate. Strain K2 cultivated in M9 medium supplemented in tryptophan grew up to an optical density at 600 nm (OD₆₀₀ₙₘ) at 6.3 and produced 311 mg/l of anthranilic acid in 31 h.

Strain K3 cultivated in M9 medium without tryptophan grew up to OD₆₀₀ₙₘ at 6.8 and produced 375 mg/l of anthranilic acid in 31 h. Strain K3 is thus a bacterial strain which is able to accumulate and secrete anthranilate without tryptophan auxotrophy.

### Example 2

Material and Methods

Culture Conditions—2 L-Fed-Batch Fermentation

Fed-batch fermentations were conducted in 2 L-bioreactor Sartorius® Stedim Biostat C containing 1.35 L of M9 culture medium supplemented by 30 g/L of glucose, 1M of IPTG and 50 μg/L of chloramphenicol. Cells were inoculated from a cryostock (strains conserved at −80° C. in the presence of 20% glycerol), 100 L into 100 mL of LB medium 1M of IPTG and 50 g/L of chloramphenicol in 1 L-baffled flask and were cultivated in a rotary shaker at 37° C. and 200 rpm, during 8 hours. Then, a 2 L-baffled flask, containing 200 mL of M9 medium supplemented with by 30 g/L of glucose, 1M of IPTG and 50 g/L of chloramphenicol, was inoculated with 2 mL aliquot of the seed culture. The cells were cultured at 37° C. and 200 rpm until OD600 reached ˜4, and were transferred into the fermenter to reach an initial OD600 at 0.2. The culture pH was controlled at 6.8 by automatic feeding of 17% (v/v) ammonia solution or 2M H₃PO₄, and the temperature was maintained at 37° C. The dissolved oxygen concentration (DO) was controlled at 20% of air saturation.

The biomass was monitored by measuring the OD at 600 nm. The extracellular concentration of anthranilate was determined by HPLC-UV after 48 hours of cultivation.

Culture Conditions—20 L-Fed-Batch Fermentation

Fed-batch fermentations were conducted in 20 L-bioreactor bioreactor Sartorius® Stedim Biostat C containing 16 L of M9 culture medium supplemented by 30 g/L of glucose, 1M of IPTG and 50 g/L of chloramphenicol. Cells were inoculated from a cryostock 100 L into 100 mL of LB medium 1M of IPTG and 50 g/L of chloramphenicol in 1 L-baffled flask and were cultivated in a rotary shaker at 37° C. and 200 rpm, during 8 hours. Then, 2×5 L-baffled flasks containing 2×1 L M9 medium supplemented with by 30 g/L of glucose, 1M of IPTG and 50 g/L of chloramphenicol, was inoculated with 2×50 mL of the seed culture. The cells were cultured at 37° C. and 200 rpm until OD600 reached ˜4, and were transferred into the fermenter to reach an initial OD600 at 0.2. The culture pH was controlled at 6.8 by automatic feeding of 35% (v/v) ammonia solution or 2M H₃PO₄, and the temperature was maintained at 37° C. The dissolved oxygen concentration (DO) was controlled at 20% of air saturation by automatically increasing supplying air from 0.2 to 1 vvm and changing the agitation speed up to 1800 rpm. During the culture the residual glucose concentration is maintained between 10 and 30 g/L.

The biomass was monitored by measuring the OD at 600 nm. The extracellular concentration of anthranilate was determined by HPLC-UV after 48 hours of cultivation.

Results

The anthranilate production of strain K3 in 2 L and 20 L fed-batch fermenters is presented in Table 4 below.

TABLE 4
anthranilate production of strain K3
in 2 L and 20 L fed-batch fermenters

| | 2 L fed-batch | 20 L fed-batch |
|---|---|---|
| | fermenter | fermenter |
| Anthranilate (g/L) | 2.3 | 5.2 |
| Yield anthranilate/glucose (g/g) | 0.02 | 0.02 |
| Productivity (g/L/h) | 0.07 | 0.11 |

### Example 3

Material and Methods

Strain Construction

Strain K2 disclosed in example 1 was engineered to replace the native promoter of the tktA gene encoding a transketolase enzyme by a stronger promoter in order to overexpress endogenous tktA enzyme, leading to strain K51.

Strain K51 was then further engineered (i) to replace the native promoter of the aroG gene encoding a 3-deoxy-D-arabino-heptulosonate-7-phosphate synthase enzyme by a stronger promoter and (ii) to mute the endogenous gene aroG directly on chromosome by introducing the mutation leading to a feedback resistant aroG enzyme. For this, aroG gene from E. coli was amplified and cloned into a plasmid by restriction/ligation method. A mutagenesis by QuickChange method was then carried out on this plasmid to mute aroG to aroG^{fbr} (G436A). Eventually, the replacement promoter was inserted in front of aroG^{fbr} gene by PCR and cloned into a plasmid allowing recombination with chromosome. This recombinant cassette comprising aroG^{fbr} gene under the control of a promoter stronger than the native promoter was then introduced in K51 strain leading to strain K77. Strain K77 was then transformed by p002 plasmid, leading to K91 strain.

Culture Conditions

K91 strains were cultured in 5 ml of LB medium during 8 h, then overnight in 10 ml of mineral medium M9 (the composition of the medium is described in table 3). 20 mg/l of tryptophan were added in K2 culture. 100 μl of each preculture were used to inoculate 25 ml of mineral medium M9 in the same conditions as preculture. The strains were then cultured during 31 h at 37° C.

Analysis

The quantity of anthranilate in the supernatant of the cultures was measured using an LC-UV method. The HPLC instrument was equipped with an column (Waters Acquity BEH C18 (50*2.1 mm; 1.7 μm)) and coupled to a UV detector: [190-400 nm]. Solution of H2O/ACN was used as a mobile phase at 40° C. at flow rate of 0.5 ml/, a linear elution gradient from 5% to 100% ACN over 8 min and an UV detector (O210 nm).

Results

Strain K91 cultivated in M9 medium without tryptophan grew up to OD₆₀₀ₙₘ at 7.0 and produced 573 mg/l of anthranilic acid in 32 h. Strain K91 is thus a bacterial strain which is able to accumulate and secrete anthranilate without tryptophan auxotrophy.

1-16. (canceled)

17. A method for producing anthranilic acid comprising culturing a recombinant bacterium and optionally, recovering anthranilic acid, wherein the non-modified bacterium corresponding to said recombinant bacterium expresses a bifunctional protein exhibiting TrpG and TrpD activities and the recombinant bacterium has been genetically modified to separately express (i) a polypeptide which exhibits glutamine amidotransferase activity (TrpG) and does not exhibit anthranilate phosphoribosyltransferase activity, and (ii) a polypeptide which exhibits anthranilate phosphoribosyltransferase activity (TrpD) and does not exhibit glutamine amidotransferase activity, and to decrease the ratio between anthranilate phosphoribosyltransferase activity and anthranilate synthase activity by comparison to the non-modified bacterium, said recombinant bacterium being able to grow in a culture medium lacking tryptophan.

18. The method of claim 17, wherein the recombinant bacterium has been genetically modified to suppress expression of the TrpD domain of the endogenous bifunctional protein TrpGD.

19. The method of claim 18, wherein the recombinant bacterium comprises a gene encoding glutamine amidotransferase (TrpG) and a gene encoding anthranilate phosphoribosyltransferase (TrpD), under the control of the same promoter thereby expressing two distinct proteins TrpG and TrpD.

20. The method of claim 18, wherein the recombinant bacterium has been genetically modified to suppress expression of the TrpD domain of the endogenous bifunctional protein TrpGD by deleting all or part of the nucleic acid sequence encoding said domain.

21. The method of claim 17, wherein the recombinant bacterium has been genetically modified to suppress expression of the endogenous bifunctional protein TrpGD.

22. The method of claim 21, wherein the recombinant bacterium has been genetically modified to suppress expression of the endogenous bifunctional protein TrpGD by deleting all or part of the nucleic acid sequence encoding said protein.

23. The method of claim 17, wherein said bacterium is Escherichia coli.

24. The method of claim 17, wherein the recombinant bacterium comprises (i) a recombinant nucleic acid comprising a gene encoding glutamine amidotransferase (TrpG) or a first operon, under the control of a first promoter, wherein said first operon comprises at least a nucleic acid sequence encoding TrpG and optionally a nucleic acid sequence encoding anthranilate synthase (TrpE), and/or (ii) a recombinant nucleic acid comprising a gene encoding anthranilate phosphoribosyltransferase (TrpD) or a second operon, under the control of a second promoter wherein said second operon comprises at least a nucleic acid sequence encoding TrpD.

25. The method of claim 24, wherein the first promoter is a promoter that is not operably linked to a gene encoding TrpG in the non-modified bacterium and/or the second promoter is a promoter that is not operably linked to a gene encoding TrpD in the non-modified bacterium.

26. The method of claim 24, wherein the second operon further comprises a nucleic acid sequence encoding indole-3-glycerol phosphate synthase enzyme (TrpC), a nucleic acid sequence encoding phosphoribosylanthranilate isomerase enzyme (TrpF), a nucleic acid sequence encoding α subunit of tryptophan synthase enzyme (TrpA) and/or a nucleic acid sequence encoding β subunit of tryptophan synthase enzyme (TrpB).

27. The method of claim 24, wherein the first promoter is stronger than the second promoter.

28. The method of claim 17, wherein the recombinant bacterium is also genetically modified to express a gene encoding a feedback resistant 3-deoxy-D-arabino-heptulosonate-7-phosphate synthase enzyme and/or genetically modified to overexpress an endogenous gene encoding a transketolase enzyme or to express a heterologous gene encoding a transketolase enzyme.

29. The method of claim 28, wherein the recombinant bacterium is also genetically modified to express a gene encoding a feedback resistant 3-deoxy-D-arabino-heptulosonate-7-phosphate synthase enzyme and genetically modified to overexpress an endogenous gene encoding a transketolase enzyme.

30. The method of claim 17, wherein the recombinant bacterium is cultivated in a culture medium lacking tryptophan or any tryptophan source.

31. The method of claim 17, wherein the recombinant bacterium is able to produce at least 1 g/L of anthranilate when cultured in 2 L fed-batch fermenter or at least 4 g/L of anthranilate when cultured in 20 L fed-batch fermenter, during 48 h in the presence of glucose as carbon source.

32. A recombinant bacterium that expresses a bifunctional protein exhibiting TrpG and TrpD activities and that has been genetically modified to separately express (i) a polypeptide which exhibits glutamine amidotransferase activity (TrpG) and does not exhibit anthranilate phosphoribosyltransferase activity, and (ii) a polypeptide which exhibits anthranilate phosphoribosyltransferase activity (TrpD) and does not exhibit glutamine amidotransferase activity, and to decrease the ratio between anthranilate phosphoribosyltransferase activity and anthranilate synthase activity by comparison to the non-modified bacterium, said recombinant bacterium being able to grow in a culture medium lacking tryptophan.

33. The recombinant bacterium of claim 33, which is an Escherichia coli bacterium which has been genetically modified to comprise a nucleic acid sequence encoding glutamine amidotransferase (TrpG) under the control of a first promoter and a nucleic acid sequence encoding anthranilate phosphoribosyltransferase (TrpD) under the control of a second promoter.

34. The recombinant bacterium of claim 33, wherein said recombinant bacterium comprises (i) a gene encoding glutamine amidotransferase (TrpG) or a first operon, under the control of a first promoter, wherein said first operon comprises at least a nucleic acid sequence encoding TrpG and optionally a nucleic acid sequence encoding anthranilate synthase (TrpE), and (ii) a gene encoding anthranilate phosphoribosyltransferase (TrpD) or a second operon, under the control of a second promoter wherein said second operon comprises at least a nucleic acid sequence encoding TrpD, and optionally a nucleic acid sequence encoding indole-3-glycerol phosphate synthase enzyme (TrpC), a nucleic acid sequence encoding phosphoribosylanthranilate isomerase enzyme (TrpF), a nucleic acid sequence encoding α subunit of tryptophan synthase enzyme (TrpA) and/or a nucleic acid sequence encoding β subunit of tryptophan synthase enzyme (TrpB).


SEQUENCE LISTING (12 sequences)

SEQ ID NO: 1 - protein, 531 aa, Escherichia coli
MADILLLDNIDSFTYNLADQLRSNGHNVVIYRNHIPAQTLIERLATMSNPVLMLSPGPGVPSEAGCMPELLTRLRGKLPIIGICLGHQAIVEAYGGYVGQAGEILHGKASSIEHDGQAMFAGLTNPLPVARYHSLVGSNIPAGLTINAHFNGMVMAVRHDADRVCGFQFHPESILTTQGARLLEQTLAWAQQKLEPANTLQPILEKLYQAQTLSQQESHQLFSAVVRGELKPEQLAAALVSMKIRGEHPNEIAGAATALLENAAPFPRPDYLFADIVGTGGDGSNSINISTASAFVAAACGLKVAKHGNRSVSSKSGSSDLLAAFGINLDMNADKSRQALDELGVCFLFAPKYHTGFRHAMPVRQQLKTRTLFNVLGPLINPAHPPLALIGVYSPELVLPIAETLRVLGYQRAAVVHSGGMDEVSLHAPTIVAELHDGEIKSYQLTAEDFGLTPYHQEQLAGGTPEENRDILTRLLQGKGDAAHEAAVAANVAMLMRLHGHEDLQANAQTVLEVLRSGSAYDRVTALAARG

SEQ ID NO: 2 - protein, 194 aa, Bacillus subtilis
MILMIDNYDSFTYNLVQYLGELGEELVVKRNDSITIDEIEELSPDFLMISPGPCSPDEAGISLEAIKHFAGKIPIFGVCLGHQSIAQVFGGDVVRAERLMHGKTSDIEHDGKTIFEGLKNPLVATRYHSLIVKPETLPSCFTVTAQTKEGEIMAIRHNDLPIEGVQFHPESIMTSFGKEMLRNFIETYRKEVIA

SEQ ID NO: 3 - protein, 531 aa, Salmonella typhimurium
MADILLLDNIDSFTWNLADQLRTNGHNVVIYRNHIPAQTLIDRLATMKNPVLMLSPGPGVPSEAGCMPELLTRLRGKLPIIGICLGHQAIVEAYGGYVGQAGEILHGKASSIEHDGQAMFAGLANPLPVARYHSLVGSNVPAGLTINAHFNGMVMAVRHDADRVCGFQFHPESILTTQGARLLEQTLAWAQQKLEPTNTLQPILEKLYQAQTLTQQESHQLFSAVVRGELKPEQLAAALVSMKIRGEHPNEIAGAATALLENAAPFPRPEYLFADIVGTGGDGSNSINISTASAFVAAACGLKVAKHGNRSVSSKSGSSDLLAAFGINLDMNADKSRQALDELGVCFLFAPKYHTGLRHAMPVRQQLKTRTLFNVLGPLINPAHPPLALIGVYSPELVLPIAETLRVLGYQRAAVVHSGGMDEVSLHAPTIVAELHDGEIKSYQLTAEDFGLTPYHQDQLAGGTPEENRDILTRLLQGKGDAAHEAAVAANVAMLMRLHGQEDLKANAQTVLDVLRNGTAYDRVTALAARG

SEQ ID NO: 4 - protein, 189 aa, Cupriavidus necator
MLLMIDNYDSFTYNLVQYFGELGEDVRTYRNDEITIEEIEALKPDHICVSPGPCSPKEAGISVAALQHFAGKIPLLGVCLGHQAIGEAFGGKVIRAKQVMHGKVSTIETTQQGVFAGLPRHFDVTRYHSLAIERETLPDCLEITAWTPDGEIMGVRHKTLAVEGVQFHPESILSEHGHALLANFVKAPR

SEQ ID NO: 5 - protein, 208 aa, Corynebacterium glutamicum
MTHVVLIDNHDSFVYNLVDAFAVAGYKCTVFRNTVPVETILAANPDLICLSPGPGYPADAGNMMALIERTLGQIPLLGICLGYQALIEYHGGKVEPCGPVHGTTDNMILTDAGVQSPVFAGLATDVEPDHPEIPGRKVPIGRYHSLGCVVAPDGIESLGTCSSEIGDVIMAARTTDGKAIGLQFHPESVLSPTGPVILSRCVEQLLAN

SEQ ID NO: 6 - protein, 338 aa, Bacillus subtilis
MNRFLQLCVDGKTLTAGEAETLMNMMMAAEMTPSEMGGILSILAHRGETPEELAGFVKAMRAHALTVDGLPDIVDTCGTGGDGISTFNISTASAIVASAAGAKIAKHGNRSVSSKSGSADVLEELEVSIQTTPEKVKSSIETNNMGFLFAPLYHSSMKHVAGTRKELGFRTVFNLLGPLSNPLQAKRQVIGVYSVEKAGLMASALETFQPKHVMFVSSRDGLDELSITAPTDVIELKDGERREYTVSPEDFGFTNGRLEDLQVQSPKESAYLIQNIFENKSSSSALSITAFNAGAAIYTAGITASLKEGTELALETITSGGAAAQLERLKQKEEEIYA

SEQ ID NO: 7 - protein, 341 aa, Cupriavidus necator
MITPQEALTRCIEHREIFHDEMLHLMRQIMQGQISPVMAAAILTGLRVKKETIGEISAAAQVMREFANKVPVADRENFVDIVGTGGDGSHTFNISTASMFVAAAAGAKIAKHGNRGVSSKSGSADVLEALGVNIMLTPEQVGQCIEETGIGFMFAPTHHPAMKNVAPIRKEMGVRTIFNILGPLTNPADAPNILMGVFHPDLVGIQVRVMQRLGAKHAIVVYGKDGMDEVSLGAATLVGELKDGEVREYEIHPEDFGLQMISNRGLKVADATESKEMLLEALTNVPGTPREIVSLNAGTALYAANVADSVEDGIRRAREAIASGAAQEKLDQFVRATQQFK

SEQ ID NO: 8 - protein, 348 aa, Corynebacterium glutamicum
MTSPATLKVLNAYLDNPTPTLEEAIEVFTPLTVGEYDDVHIAALLATIRTRGEQFADIAGAAKAFLAAARPFPITGAGLLDSAGTGGDGANTINITTGASLIAASGGVKLVKHGNRSVSSKSGSADVLEALNIPLGLDVDRAVKWFEASNFTFLFAPAYNPAIAHVQPVRQALKFPTIFNTLGPLLSPARPERQIMGVANANHGQLIAEVFRELGRTRALVVHGAGTDEIAVHGTTLVWELKEDGTIEHYTIEPEDLGLGRYTLEDLVGGLGTENAEAMRATFAGTGPDAHRDALAASAGAMFYLNGDVDSLKDGAQKALSLLADGTTQAWLAKHEEIDYSEKESSND

SEQ ID NO: 9 - DNA, 68 bp, Artificial Sequence
  Primer 1
gcgcagcagaaactagagccagccaacacgctgcaaccgattctgtaagtgtaggctggagctgcttc

SEQ ID NO: 10 - DNA, 65 bp, Artificial Sequence
  Primer 2
aatcgccttgtctgcgacgattttcgctaaaacggtttgcatcatatgggaattagccatggtcc

SEQ ID NO: 11 - DNA, 32 bp, Artificial Sequence
  Primer 3
agtatggctagcttaccctcgtgccgccagtg

SEQ ID NO: 12 - DNA, 79 bp, Artificial Sequence
  Primer 4
gactagaagcttcctctagaaataattttgtttaactttaagaaggagaatcgatatgaacacgctgcaaccgattctg